(19) United States
(12) Patent Application Publication
(10) Pub. No.: US 2016/0206863 A1
(43) Pub. Date: Jul. 21, 2016

(54) METHOD AND APPARATUS USING MUSIC THERAPY OR RESONANT BOOK TO REMOVE ASBESTOS AND MERCURY FROM THE HUMAN BODY

(71) Applicant: Sayfone Phounsavan, Thousand Oaks (CA)

(72) Inventor: Sayfone Phounsavan, Thousand Oaks (CA)

(21) Appl. No.: 15/000,383

(22) Filed: Jan. 19, 2016

Related U.S. Application Data
(60) Provisional application No. 62/105,416, filed on Jan. 20, 2015.
(60) Provisional application No. 62/114,288, filed on Feb. 10, 2015.
(60) Provisional application No. 62/203,950, filed on Aug. 12, 2015.

(51) Int. Cl. A61M 37/00; A61H 39/04

(52) U.S. Cl. CPC A61M 37/00; A61H 39/04; A61M 2202/203; A61M 2202/206; A61H 2201/169

(57) ABSTRACT

A method and apparatus using a music therapy or a resonant book to remove or excrete asbestos from the human body is described. The music therapy comprises a set of one or more vibrational energy songs. The music therapy may further comprise (a) storing said vibrational energy songs on a music player, cell phone or computer, and (b) listening to said vibrational energy song on said music player, cell phone or computer. The person may clap hands or press corresponding acupuncture points of an internal organ on one hand or both hands while listening to the song. Each song does not contain one or more notes in the set of seven standard notes: {Do (1), Re (2), Mi (3), Fa (4), Sol (5), La (6), Si (7)}. Each note may be sung or played at a higher pitch or a lower pitch. The music therapy based method or apparatus may further comprise spraying specially processed herbal water on a selected acupuncture point before or after the music therapy, such as Conception Vessel C.V.1. The resonant book based method or apparatus may further comprise holding a resonant book (such as a book of the Bible) on hand or placing the resonant book on the human body or a selected acupuncture point (such as Conception Vessel C.V.8), with or without practicing the music therapy. Practicing any of the methods or apparatuses may further remove or excrete other toxins (such as mercury, lead, arsenic, aluminum), germs (such as viruses, bacteria, fungi), gene mutation cells (such as CD332), and cancer stem cells (such as CD24, CD44, CD133, CD29, CD152) from the human body to urine, mucus, feces, or the skin and body surface.

### CROSS REFERENCE TO RELATED APPLICATIONS

This application claims benefit under 35 U.S.C. 119(e) of U.S. Provisional Patent Application Ser. No. 62/105,416, filed Jan. 20, 2015, U.S. Provisional Patent Application Ser. No. 62/114,288, filed Feb. 10, 2015 and U.S. Provisional Patent Application Ser. No. 62/203,950, filed Aug. 12, 2015, the entire contents which are incorporated herein by reference.

### FIELD OF THE INVENTION

The invention generally relates to the field of asbestos and mercury diagnosis and removal for cancer prevention and treatment. Specifically, the invention relates to the removal or excretion of asbestos and mercury from the human body.

### BACKGROUND

Asbestos is a group of minerals each composed of millions of microscopic fibers that can be released by abrasion and other processes. These fibers are resistant to water, heat, fire, and chemicals. They also do not conduct electricity. With these unique properties, asbestos has been used widely in the construction and automotive industries and for thermal insulation at homes.

The United States Environmental Protection Agency (EPA) defines six mineral types as asbestos including those belonging to the serpentine class and those belonging to the amphibole class. All six asbestos mineral types are known to be human carcinogens. They are commonly known by their colors, as “blue asbestos”, “brown asbestos”, “white asbestos”, and so on.

If products containing asbestos are disturbed, the tiny fibers are released into the air. When they are breathed in, they can become trapped in the lungs and stay there for many years. Over time these fibers can accumulate and lead to serious health problems, including asbestosis, lung cancer, and mesothelioma.

Asbestosis is an aggressive pulmonary disease that can be challenging to diagnose since symptoms resemble those of less serious conditions like asthma and pneumonia. More than 1,400 asbestosis-related deaths were recorded in the United States in 2005. Mesothelioma is a rare cancer that affects the lining of the lungs, chest cavity, or abdomen. Studies have also suggested an association between asbestos exposure and other cancers, including cancers of the gastrointestinal tract, throat, kidney, brain, urinary bladder, voice box, and gall bladder.

A study conducted by Omura (2012) has shown food and water sources can also contain asbestos. Because asbestos is not water soluble, standard methods of detecting asbestos include either using high microscopic optical magnification or electroscope. Even simple detection of asbestos in drinking water requires a sample of at least 100 cc, and usually requires one liter of water. EPA requires that when the length of asbestos crystal is over 10 microns, it need to be reported. The problem is by the time asbestos in drinking water or bottled water is brought to a standard laboratory, most of the large crystals longer than 10 microns have been already deposited on the bottom of the water bottle. Most of asbestos crystals that can be found are usually less than 2 microns. Particularly, most of them are less than 0.5 microns, which is generally considered and grouped as dust. Therefore, if any water contains asbestos crystals of less than 2 microns, particularly, less than 0.5 microns, most of the water will erroneously be considered as non-asbestos containing water.

Mercury (chemical symbol Hg) is another source of toxins, other than asbestos, which may be present in the human body. Mercury is a heavy metal occurring in several forms (elementary mercury, inorganic mercury and organic mercury), which can produce toxicity or cause death with less than a gram. Humans, unfortunately, can hardly avoid exposure to mercury. The sources are mostly from fish consumption, mercury vapor in mercury amalgam dental restorations, and environmental contamination that emits mercury (such as from coal-fired power plants and volcanos), to name a few. Very often, the amount is so minute that a normal person would not have noticed any symptom. Only when the mercury concentration in the body exceeds an acceptable level, can it bring long-term damage to the body. According to the United States Environmental Protection Agency (EPA) report, when the amount of mercury in the mouth exceeds 36 parts per trillion (ppt), or 300 ng/m³, it is high enough to damage the brain and nerve cells directly.

Mercury detection apparatuses are usually costly and may require long and arduous procedures to perform; they are often only available in large hospitals and medical centers and they check through different samples such as blood, urine, hair, etc. The Mercury Detector RA 195, developed by St. Petersburg State University in Russia, is one of the world's most sensitive, accurate, and trustworthy apparatuses, but its detection ability still has a limit, in the range of 10⁻¹².

Omura developed a Bi-Digital O-Ring Test (BDORT) technique for medical diagnosis by imaging an internal organ of a patient against a sample tissue holding on a hand in 1993. See the U.S. Pat. No. 5,188,107. The technique first determines which finger can be best used with thumb to form an O-ring shape. After finding the finger, an O-ring shape is formed. The sample tissue of an internal organ corresponding to the organ to be imaged is placed on or in the close vicinity of the patient's other hand. The sample tissue may be a microscopic slide. If the patient has a particular illness (such as cancer) or substance deposit in the body (such as asbestos or mercury), which is the same type as that of the sample tissue on the patient's hand, then the O-ring technique will cause his thumb and finger to be parted and accordingly the illness area can be imaged. Because BDORT is based on the electromagnetic field (EMF) resonance phenomenon between two identical molecules, it can measure any substance including asbestos even in small samples (less than 0.5 cc) noninvasively. Diagnosis of asbestos presence or absence and measurement of the amount of asbestos in the human body has since become quite simple.

There are prior art methods that have been proposed to remove asbestos from the human body (Omura 2012). One method is to take cilantro. By boiling cilantro leaves in water for about 10 minutes and then eating the leaves and drinking the water can excrete asbestos inside of the body into urine. At the same time, other toxins (such as mercury, lead, arsenic, and aluminum) are also excreted.

Another method is to increase normal cell telomere levels (Omura 2012). A telomere is a region of repetitive DNA at an end of a chromosome, which protects the end of the chromosome from deterioration or from fusion with neighboring chromosomes. When telomeres in normal cells are in dysfunction, it can cause gene mutations which may lead to cancer when untreated in advance. Omura discovers by increasing normal cell telomere levels of over 500-550 ng BDORT units, excretion of asbestos, mercury, lead, arsenic, and aluminum in the urine can be significantly increased. Nutrients such as Astragalus and Boswellia serrata, certain fruits such as bitter melon, mango, papaya, and pineapple, as well as certain fishes such as tuna, salmon, and eel, can increase normal cell telomere levels, while markedly decreasing the cancer cell telomere levels. Inserting a press needle on a special acupuncture point and mechanically simulating the press and release procedure 250 times can also help.

These prior art methods have proved efficacy of excreting asbestos and other toxins from the human body, but they centered on taking foods or nutrients, or practicing an acupuncture procedure. In an embodiment of the invention, a music therapy or resonant book based method or apparatus is proposed to remove or excrete asbestos and other toxins from the human body. The music therapy may include one or more vibrational energy songs each targeting people belong to one of the Five Elements. The resonant book may include a book (such as a book of the Bible) which can cause resonance with the human body when using a resonance-based test technique (such as BDORT).

The Five Elements are Metal, Water, Wood, Fire, and Earth. The Elements are representations of the transformation that occurs in the world around humans; they are metaphors for describing how things interact and relate with each other.

According to Schiesser (2016), metal type people like minimalism. They are organized, clean, and contained. They can be controlling, ambitious, forceful and set in their ways as metal is very strong; and they are self-reliant and prefer to handle their problems alone. The negative emotion associated with metal is grief, while the positive emotion is courage.

Water type people appear a bit reserved, yet are often very creative, sometimes even eccentric. They can appear cool and stoic, yet have the capacities to be still and deeply reflect. The negative emotion associated with water is fear, while the positive emotion is calmness.

Wood type people are often aggressive or assertive, direct, and can have a strong temper and a lot of drive. They are usually outgoing and socially conscious and can be insensitive. The Wood element is associated with negative feelings of anger, and positive feelings of patience and altruism.

Fire type people are charming, fun, mischievous, easily excitable, and change emotional states rapidly. They love change, bright colors and environments that stimulate. The negative emotion is hate, while its positive emotion is joy.

Earth type people are usually warm, kind and supportive. They can be overprotective and tend to merge with their environment, having difficulties with boundaries. The negative emotion of the Earth element is worry and its positive emotion is empathy.

While each element type people have their own personalities, they may have a mix of personalities with other element type people.

### SUMMARY OF THE INVENTION

A first preferred invention encompasses using a resonance-based test technique, such as the BDORT technique, to identify which type of the Five Elements a person may belong to. The Five Elements are Metal, Water, Wood, Fire, and Earth. A color sample (such as a red cloth, a yellow cloth, a white cloth, a black cloth, or a green cloth) is used for the test.

A second preferred invention further encompasses a method or apparatus using a music therapy or resonant book to remove or excrete asbestos from the human body to urine (through urination), mucus (through coughing), or feces.

A third preferred invention further encompasses a method or apparatus using a herbal treatment procedure to remove or excrete mercury from the skin and body surface or from the human body to urine (through urination), mucus (through coughing), or feces.

It should be noted that in each preferred invention or embodiment described in the specification for asbestos or mercury removal or excretion, the invention or embodiment may further comprise removing or excreting other toxins, germs, gene mutation cells, and cancer stem cells when present in the human body to urine, mucus, feces, or the skin and body surface. The toxins may include asbestos, mercury (Hg), lead (Pb), arsenic (As), and aluminum (Al). The germs may include viruses, bacteria, and fungi. The gene mutation cells may include CD332 (Cluster of Differentiation 332). The cancer stem cells may include CD24, CD44, CD133, CD29, and CD152.

The music therapy comprises one or more vibrational energy songs each targeting people belong to one of the Five Elements. The music therapy is developed based on Yellow Emperor's Inner Canon, the earliest written text on Traditional Chinese Medicine. All vibrational energy songs for music therapy are created to obey the theory.

It should be further noted that while each vibrational energy song given in the following embodiments and figure descriptions may be proposed to target a person belonging to one of the Five Elements, the song can target any person, regardless of whether the person knows his or her element type or not.

The resonant book, referred to as an optimal-dose resonant book, may comprise one or more books which can cause resonance with the human body when using a resonance-based test technique (such as BDORT). The book may comprise a religious, science or medicine book (such as a book of the Bible).

In a first embodiment of the invention, a resonance-based test technique, such as the BDORT technique, may be used identify which element type the person belongs to. The method may require assistance of a second person. The method comprises (1) determining which finger of the person can be best used with thumb to form an O-ring shape, (2) asking the person to watch a color sample, and (3) testing if the formed O-ring between the finger and thumb can be pulled off and opened by a second person. The color sample may be a red cloth, a yellow cloth, a white cloth, a black cloth, or a green cloth. When the O-ring can be pulled off and opened by the second person and if the color sample is red, the person belongs to a Metal type. When the O-ring can be pulled off and opened by the second person and if the color sample is yellow, the person belongs to a Water type. When the O-ring can be pulled off and opened by the second person and if the color sample is white, the person belongs to a Wood type. When the O-ring can be pulled off and opened by the second person and if the color sample is black, the person belongs to a Fire type. When the O-ring can be pulled off and opened by the second person and if the color sample is green, the person belongs to an Earth type.

In a second embodiment of the invention, the music therapy may comprise a vibrational energy song to remove or excrete asbestos from the human body. The vibrational energy song may target a person belonging to one of the Five Elements. The method comprises (1) singing on lips or inside the mind of the person, (2) listening to a vibrational energy song on a music player, cell phone or computer, or (3) singing and listening to the song at the same time. The person may clap hands or press corresponding acupuncture points of an internal organ on one hand or both hands while singing or listening to the song.

In a third embodiment of the invention, the vibrational energy song may further comprise three or more music notes. Each song targeting an element type may not contain one or more notes in the set of seven standard notes: {Do (1), Re (2), Mi (3), Fa (4), Sol (5), La (6), Si (7)}. Each note may be sung or played at a higher pitch or a lower pitch. For example, a vibrational energy song for Metal type people may not contain the La (6) or Sol (5) note. The vibrational energy song for Water type people may not contain the Do (1) note. The vibrational energy song for Wood type people may not contain the Re (2) note. The vibrational energy song for Fire type people may not contain the Mi (3) or La (6) note. The vibrational energy song for Earth type people may not contain the Sol (5) or Mi (3) note.

In a fourth embodiment of the invention, the vibrational energy song for Metal type people may comprise the following notes: {| 1+2+3+| 3+5 3 | 3 1 2 | 2 3 5 | 5 3 1+|}, where 1+ is one pitch higher than Do (1), 2+ is one pitch higher than Re (2), 3+ is one pitch higher than Mi (3), and “| ” indicates the end of the previous note or syllable. The vibrational energy song for Water type people may comprise the following notes: {| 2 3 5 6 | 6 3+2+3+| 3+5 6 3+| 3+2+3+5 | 5 5 6 3 |}. The vibrational energy song for Wood type people may comprise the following notes: {| 1 3 5 6 | 6 3+1+3+| 3+5 6 3 | 3 1 3 5 | 5 6 3 1+ |}. The vibrational energy song for Fire type people may comprise the following notes: {| 1 2 5 6 | 6 1+2+5 | 5 6 1+2+ | 2+5 6 1+ |}. The vibrational energy song for Earth type people may comprise the following notes: {| 1 2 3 | 3 6 3+| 3+1+2+| 2+3+6 | 6 3+1+|}.

Alternatively, the vibrational energy song for Metal type people may comprise the following notes: {| 6++3+++6++| 1+++2++6+| 6+3+6+| 1+2+6 | 6 3 6 |}. The vibrational energy song for Water type people may comprise the following notes: {| 3++5++6++ | 6++2+++6++ | 3++5+6+ | 6+2+6+| 3 5 6 |}. The vibrational energy song for Wood type people may comprise the following notes: {| 6 1+6 | 6+3+5+| 6+1++6+| 6++3++5++ | 6++1++1+++ |}. The vibrational energy song for Fire type people may comprise the following notes: {| 1 2 3 | 3 5 1+| 1+2+3+| 3+5+1++| 1++2++3++ |}. The vibrational energy song for Earth type people may comprise the following notes: {| 1 2 6−| 6 5+6+| 1++2++6+| 6++5++6++| 1+++2+++6++ |}. In each vibrational energy song, “| ” indicates the end of the previous note or syllable. Any note N (where N represents any note, for example, note 1, 2, 3, 5 or 6) is one pitch higher than N−; N+ is one pitch higher than N; N++ is one pitch higher than N+; and N+++ is one pitch higher than N++.

In a fifth embodiment of the invention, the music therapy may comprise a set of one or more vibrational energy songs targeting a person of any element type, as long as the set includes a vibrational energy song for the element type the person belongs to. Each vibrational energy song that targets people belonging to one of the Five Elements may further comprise three or more notes solely using at most six of the following seven music notes: {Do (1), Re (2), Mi (3), Fa (4), Sol (5), La (6), Si (7)}. The music therapy may further comprise appending {| 1+2+5 6 |} to the end of the set of vibrational energy songs before repeating the music therapy again, where 1+ is one pitch higher than Do (1), 2+ is one pitch higher than Re (2), and “| ” indicates the end of the previous note or syllable. The set of vibrational energy songs may be placed in a select order. A preferred embodiment may further comprise repeating the music therapy as many times as is desired at any time.

In a sixth embodiment of the invention, the music therapy may further comprise practicing a Music Therapy 1-2-3 Pathway procedure to remove or excrete asbestos from the human body. The procedure may comprise using a Washing Therapy, an Aromatherapy, and/or an Elimination Therapy.

In a seventh embodiment of the invention, the music therapy may further comprise practicing a Hand and Foot Wash procedure to remove or excrete mercury from the human body. The procedure may comprise using one hand to scoop up specially processed herbal water, such as Deegua Root water, in a washbasin to wash the other hand and forearm for 40 or more cycles. The procedure may further comprise using one hand to scoop up the specially processed herbal water to wash each foot in the washbasin for 40 or more cycles.

In an eighth embodiment of the invention, the music therapy may further comprise practicing a first Herbal Water Spray procedure to remove or excrete mercury from any of the 6 or more Yin-Yang Acupuncture Points in the human body. The Yin-Yang Acupuncture Points are present in the six layers in the meridian system of the human body. The specially processed herbal water may be any kind of sulfhydryl group (SH)-containing herbs that exhibit high binding affinity of mercury, such as SF 93 Meridian water. The six outermost to innermost layers in the meridian system are Tai-Yang Meridian (associated with skin and body surface), Yang-Ming Meridian (associated with the gastrointestinal tract, or GI tract), Shao-Yang Meridian (associated with Lymphatic system), Tai-Yin Meridian (associated with spleen and lung), Shao-Yin Meridian (associated with heart and kidney), and Jue-Yin Meridian (associated with brain and liver). The locations of the meridian system (comprising 12 standard meridians and 8 extraordinary vessels) may be found in any Acupuncture textbook. The procedure may comprise spraying the specially processed herbal water on one or more Yin-Yang Acupuncture Points in a selected meridian and using additional methods and apparatuses to remove mercury from the six layers in the meridian system.

In a ninth embodiment of the invention, the music therapy may further comprise practicing a second Herbal Water Spray procedure to remove or excrete mercury from any of the 10 Inherited Acupuncture Points in the human body. The specially processed herbal water may be any kind of sulfhydryl group (SH)-containing herbs that exhibit high binding affinity of mercury, such as SF 93 Meridian water. The procedure may comprise spraying the specially processed herbal water on one or more Inherited Death Acupuncture Points in the human body. The location of the Inherited Death Acupuncture Point is inherited from a parent of the person. These Inherited Death Acupuncture Points may be found in any Acupuncture textbook.

In a tenth embodiment of the invention, the music therapy may further comprise practicing a third Herbal Water Spray procedure to remove or excrete mercury from any of the 16 or more Acquired Acupuncture Points in the human body. The specially processed herbal water may be any kind of sulfhydryl group (SH)-containing herbs that exhibit high binding affinity of mercury, such as SF 93 Meridian water. The procedure may comprise spraying the specially processed herbal water on 16 or more Acquired Death Acupuncture Points in the human body. These 16 or more Acquired Death Acupuncture Points may be found in any Acupuncture textbook.

In an eleventh embodiment of the invention, a music therapy based method or apparatus may be used to implement each of the above relevant preferred embodiments. Each vibrational energy song may be stored on a music player, cell phone, or computer. Each method or apparatus may further comprise clapping hands or pressing corresponding acupuncture points of an internal organ on one hand or both hands during the music therapy. The method or apparatus may further comprise spraying specially processed herbal water on a selected acupuncture point before or after the music therapy. The selected acupuncture point may be Conception Vessel C.V.1. It may further comprise reducing insomnia. These acupuncture points may be found in any Acupuncture textbook.

In a twelfth embodiment of the invention, a resonant book based method or apparatus may be used to further remove asbestos from the human body to urine (through urination), mucus (through coughing), or feces. The method or apparatus may comprise holding a resonant book on hand or placing the resonant book on a selected acupuncture point or on the human body, with or without practicing the music therapy. The resonant book, referred to as an optimal-dose resonant book, may comprise one or more books which can cause resonance with the human body when using a resonance-based test technique (such as BDORT). The book may comprise a religious, science or medicine book (such as a book of the Bible). The selected acupuncture point may be Conception Vessel C.V.8.

### THE BRIEF DESCRIPTION OF DRAWINGS

FIG. 1 shows a method of using a Bi-Digital O-Ring Test (BDORT) technique to identify an element type a person belongs to, in accordance with the invention;

FIG. 2 shows a method of using a music therapy to remove asbestos from the human body, in accordance with the invention;

FIG. 3A shows a first embodiment of five vibrational energy songs each targeting people belonging to one of the Five Elements during a music therapy, in accordance with the invention;

FIG. 3B shows a second embodiment of five vibrational energy songs each targeting people belonging to one of the Five Elements during a music therapy, in accordance with the invention;

FIG. 4A shows a first embodiment of a vibrational energy song for Metal type people during a music therapy, in accordance with the invention;

FIG. 4B shows a second embodiment of a vibrational energy song for Metal type people during a music therapy, in accordance with the invention;

FIG. 5A shows a first embodiment of a vibrational energy song for Water type people during a music therapy, in accordance with the invention;

FIG. 5B shows a second embodiment of a vibrational energy song for Water type people during a music therapy, in accordance with the invention;

FIG. 6A shows a first embodiment of a vibrational energy song for Wood type people during a music therapy, in accordance with the invention;

FIG. 6B shows a second embodiment of a vibrational energy song for Wood type people during a music therapy, in accordance with the invention;

FIG. 7A shows a first embodiment of a vibrational energy song for Fire type people during a music therapy, in accordance with the invention;

FIG. 7B shows a second embodiment of a vibrational energy song for Fire type people during a music therapy, in accordance with the invention;

FIG. 8A shows a first embodiment of a vibrational energy song for Earth type people during a music therapy, in accordance with the invention;

FIG. 8B shows a second embodiment of a vibrational energy song for Earth type people during a music therapy, in accordance with the invention;

FIG. 9 shows an embodiment of a set of five vibrational energy songs targeting a person of any element type during a music therapy, in accordance with the invention;

FIG. 10 shows an embodiment of practicing a Music Therapy 1-2-3 Pathway procedure to remove asbestos from the human body after a music therapy, in accordance with the invention;

FIGS. 11A to 11B show an embodiment of practicing a Hand and Foot Wash procedure to remove mercury from the human body before, after or during a music therapy, in accordance with the invention;

FIG. 12 shows an embodiment of practicing a first Herbal Water Spray procedure to remove mercury from any of the 6 or more Yin-Yang Acupuncture Points in the six layers in the meridian system of the human body before, after or during a music therapy, in accordance with the invention;

FIG. 13 shows an embodiment of practicing a second Herbal Water Spray procedure to remove mercury from any of the 10 Inherited Death Acupuncture Points in the human body before, after or during a music therapy, in accordance with the invention;

FIGS. 14A and 14B show an embodiment of practicing a third Herbal Water Spray procedure to remove mercury from any of the 16 or more Acquired Death Acupuncture Points in the human body before, after or during a music therapy, in accordance with the invention; and

FIG. 15 shows an embodiment of using a resonant book to remove or excrete asbestos from the human body, in accordance with the invention.

### DETAILED DESCRIPTION OF THE INVENTION

The following description is presently contemplated as the best mode of carrying out the invention. This description is not to be taken in a limiting sense but is made merely for the purpose of describing the principles of the invention. The scope of the invention should be determined by referring to the appended claims.

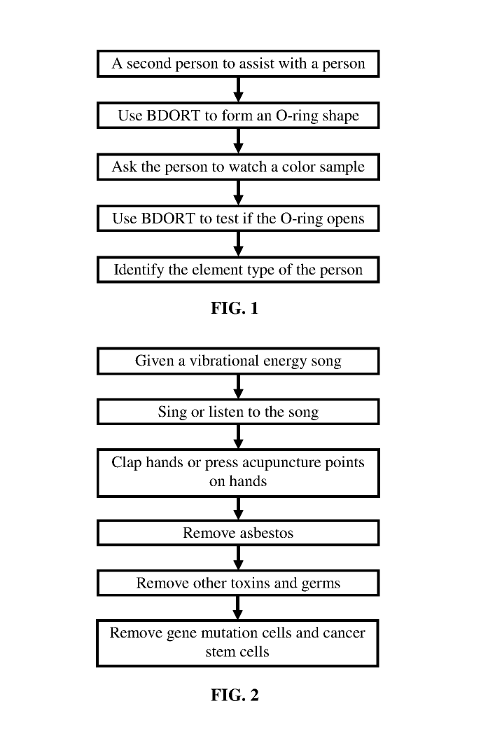

FIG. 1 shows a method of using a Bi-Digital O-Ring Test (BDORT) technique to identify an element type a person belongs to, in accordance with the invention. The method may require assistance of a second person. The method comprises (1) determining which finger of the person can be best used with thumb to form an O-ring shape, (2) asking the person to watch a color sample, and (3) testing if the formed O-ring between the finger and thumb can be pulled off and opened by a second person. The color sample may be a red cloth, a yellow cloth, a white cloth, a black cloth, or a green cloth. When the O-ring can be pulled off and opened by the second person and if the color sample is red, the person belongs to a Metal type. When the O-ring can be pulled off and opened by the second person and if the color sample is yellow, the person belongs to a Water type. When the O-ring can be pulled off and opened by the second person and if the color sample is white, the person belongs to a Wood type. When the O-ring can be pulled off and opened by the second person and if the color sample is black, the person belongs to a Fire type. When the O-ring can be pulled off and opened by the second person and if the color sample is green, the person belongs to an Earth type.

FIG. 2 shows a method of using a music therapy to remove asbestos from the human body, in accordance with the invention. The music therapy may comprise one or more vibrational energy songs. Each vibrational energy song targets a person with one of the Five Elements. The Five Elements are Metal, Water, Wood, Fire, and Earth. The method comprises (1) singing on lips or inside the mind of the person, (2) listening to a vibrational energy song on a music player, cell phone or computer, or (3) singing and listening to the song at the same time. The person may clap hands or press corresponding acupuncture points of an internal organ on one hand or both hands while singing or listening to the song. The method may also comprise removing or excreting other toxins, germs, gene mutation cells, and cancer stem cells from the human body to urine, mucus, feces, or the skin and body surface.

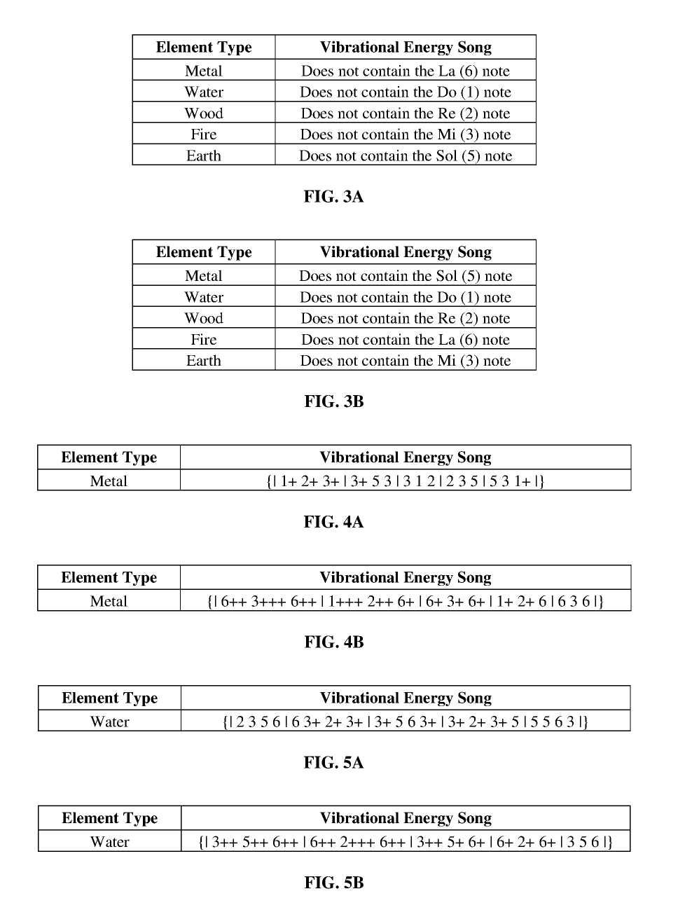

FIG. 3A shows a first embodiment of five vibrational energy songs each targeting people belonging to one of the Five Elements, in accordance with the invention. The Five Elements are Metal, Water, Wood, Fire, and Earth. The vibrational energy song for Metal type people may not contain the La (6) note. The vibrational energy song for Water type people may not contain the Do (1) note. The vibrational energy song for Wood type people may not contain the Re (2) note. The vibrational energy song for Fire type people may not contain the Mi (3) note. The vibrational energy song for Earth type people may not contain the Sol (5) note. Each note may be further sung or played at a higher pitch or a lower pitch.

FIG. 3B shows a second embodiment of five vibrational energy songs each targeting people belonging to one of the Five Elements, in accordance with the invention. The Five Elements are Metal, Water, Wood, Fire, and Earth. The vibrational energy song for Metal type people may not contain the Sol (5) note. The vibrational energy song for Water type people may not contain the Do (1) note. The vibrational energy song for Wood type people may not contain the Re (2) note. The vibrational energy song for Fire type people may not contain the La (6) note. The vibrational energy song for Earth type people may not contain the Mi (3) note. Each note may be further sung or played at a higher pitch or a lower pitch.

FIG. 4A shows a first embodiment of a vibrational energy song for Metal type people, in accordance with the invention. The vibrational energy song for Metal type people may comprise the following notes: {| 1+2+3+| 3+5 3 | 3 1 2 | 2 3 5 | 5 3 1+|}, where 1+ is one pitch higher than Do (1), 2+ is one pitch higher than Re (2), 3+ is one pitch higher than Mi (3), and “|” indicates the end of the previous note or syllable. Each note may be further sung or played at a higher pitch or a lower pitch.

FIG. 4B shows a second embodiment of a vibrational energy song for Metal type people, in accordance with the invention. The vibrational energy song for Metal type people may comprise the following notes: {| 6++3+++6++ | 1+++2++6+ | 6+3+6+ | 1+2+6 | 6 3 6 |}, where a note N+ is one pitch higher than N; N++ is one pitch higher than N+; and N+++ is one pitch higher than N++, and “|” indicates the end of the previous note or syllable. Each note may be further sung or played at a higher pitch or a lower pitch.

FIG. 5A shows a first embodiment of a vibrational energy song for Water type people, in accordance with the invention. The vibrational energy song for Water type people may comprise the following notes: {| 6++3+++6++| 1+++2++6+| 6+3+6+| 1+2+6 | 6 3 6 |}, where 2+ is one pitch higher than Re (2), 3+ is one pitch higher than Mi (3), and “|” indicates the end of the previous note or syllable. Each note may be further sung or played at a higher pitch or a lower pitch.

FIG. 5B shows a second embodiment of a vibrational energy song for Water type people, in accordance with the invention. The vibrational energy song for Water type people may comprise the following notes: {| 3++5++6++| 6++2+++6++ | 3++5+6+ | 6+2+6+ | 3 5 6 |}, where a note N+ is one pitch higher than N; N++ is one pitch higher than N+; and N+++ is one pitch higher than N++, and “|” indicates the end of the previous note or syllable. Each note may be further sung or played at a higher pitch or a lower pitch.

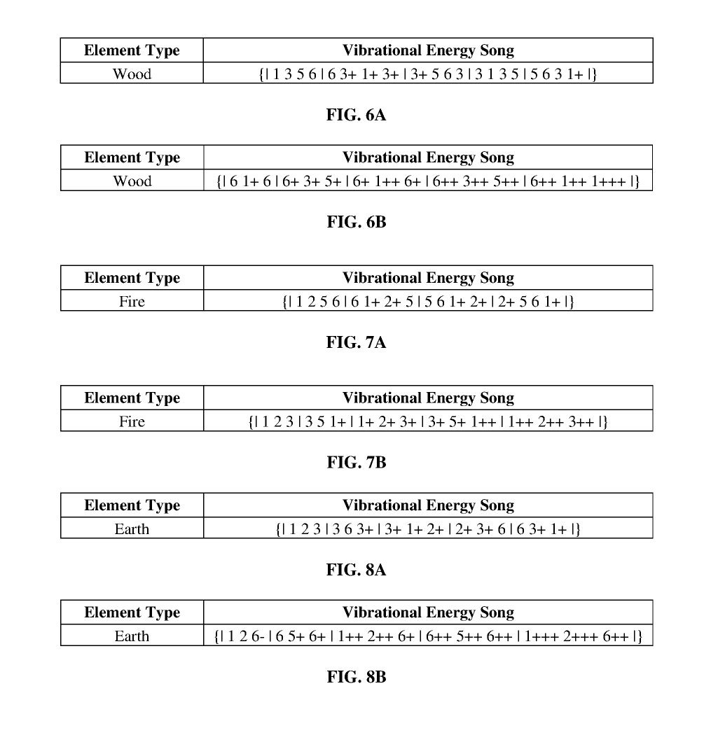

FIG. 6A shows a first embodiment of a vibrational energy song for Wood type people, in accordance with the invention. The vibrational energy song for Wood type people may comprise the following notes: {| 1 3 5 6 | 6 3+1+3+| 3+5 6 3 | 3 1 3 5 | 5 6 3 1+ |}, where 1+ is one pitch higher than Do (1), 3+ is one pitch higher than Mi (3), and “| ” indicates the end of the previous note or syllable. Each note may be further sung or played at a higher pitch or a lower pitch.

FIG. 6B shows a second embodiment of a vibrational energy song for Wood type people, in accordance with the invention. The vibrational energy song for Wood type people may comprise the following notes: {| 6 1+6 | 6+3+5+| 6+1++6+ | 6++3++5++| 6++1++1+++|}, where a note N+ is one pitch higher than N; N++ is one pitch higher than N+; and N+++ is one pitch higher than N++, and “| ” indicates the end of the previous note or syllable. Each note may be further sung or played at a higher pitch or a lower pitch.

FIG. 7A shows a first embodiment of a vibrational energy song for Fire type people, in accordance with the invention. The vibrational energy song for Fire type people may comprise the following notes: {| 1 2 5 6 | 6 1+2+5 | 5 6 1+2+ | 2+5 6 1+ |}, where 1+is one pitch higher than Do (1), 2+ is one pitch higher than Re (2), and “|” indicates the end of the previous note or syllable. Each note may be further sung or played at a higher pitch or a lower pitch.

FIG. 7B shows a second embodiment of a vibrational energy song for Fire type people, in accordance with the invention. The vibrational energy song for Fire type people may comprise the following notes: {| 1 2 3 | 3 5 1+| 1+2+3+ | 3+5+1++| 1++2++3++|}, where a note N+ is one pitch higher than N; and N++ is one pitch higher than N+, and “|” indicates the end of the previous note or syllable. Each note may be further sung or played at a higher pitch or a lower pitch.

FIG. 8A shows a first embodiment of a vibrational energy song for Earth type people, in accordance with the invention. The vibrational energy song for Earth type people may comprise the following notes: {| 1 2 3 | 3 6 3+| 3+1+2+| 2+3+6 | 6 3+1+|}, where 1+ is one pitch higher than Do (1), 2+ is one pitch higher than Re (2), 3+ is one pitch higher than Mi (3), and “| ” indicates the end of the previous note or syllable. Each note may be further sung or played at a higher pitch or a lower pitch.

FIG. 8B shows a second embodiment of a vibrational energy song for Earth type people, in accordance with the invention. The vibrational energy song for Earth type people may comprise the following notes: {| 1 2 6− | 6 5+6+ | 1++2++6+ | 6++5++6++| 1+++2+++6++|}, where a note N is one pitch higher than N−; N+ is one pitch higher than N; N++ is one pitch higher than N+; and N+++ is one pitch higher than N++, and “|” indicates the end of the previous note or syllable. Each note may be further sung or played at a higher pitch or a lower pitch.

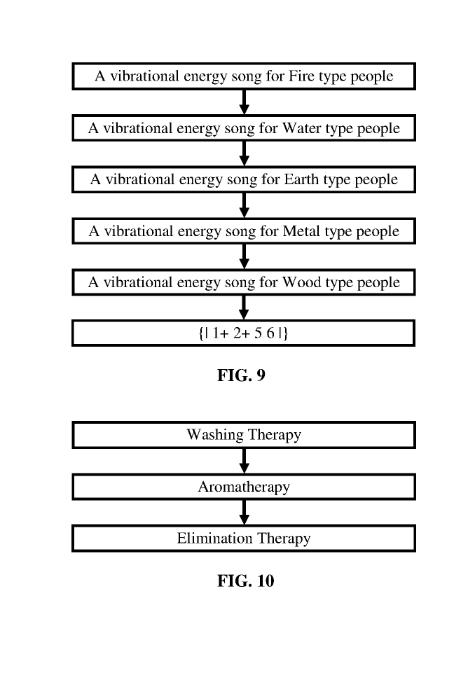

FIG. 9 shows an embodiment of a set of five vibrational energy songs targeting a person of any element type during a music therapy, in accordance with the invention. The figure may include a vibrational energy song for Fire type people, a vibrational energy song for Water type people, a vibrational energy song for Earth type people, a vibrational energy song for Metal type people, and a vibrational energy song for Wood type people. The music therapy may further comprise one or more vibrational energy songs as long as the set includes a vibrational energy song for the element type the person belongs to. The element type is a Metal element, a Water element, a Wood element, a Fire element, or an Earth element. The music therapy may further comprise appending {| 1+2+5 6 |} to the end of the set of vibrational energy songs before repeating the music therapy again, where 1+ is one pitch higher than Do (1), 2+ is one pitch higher than Re (2), and “| ” indicates the end of the previous note or syllable. The set of one or more vibrational energy songs may be placed in a select order. A preferred embodiment may further comprise repeating the music therapy as many times as is desired at any time.

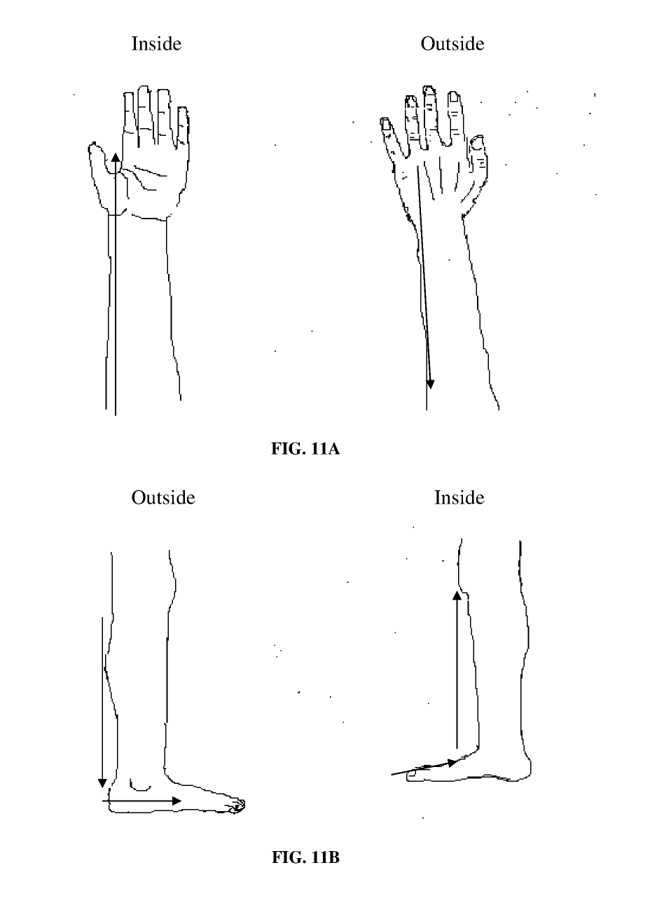

FIG. 10 shows an embodiment of practicing a Music Therapy 1-2-3 Pathway procedure to remove asbestos from the human body after a music therapy, in accordance with the invention. The Music Therapy 1-2-3 Pathway procedure may comprise the following one or more steps:

Step 1: (Washing Therapy) Wash both hands with specially processed herbal water, such as Laotian specialized Evian water, for 25 or more times, each time with 0.2 cc water (a capful of herbal water may be enough for 5 times). Simultaneously wash both sides of the hands and massage the illness related acupuncture points on both hands as needed. The locations of illness related acupuncture points may be found in (Omura 2012).

Step 2: (Aromatherapy) Smell selected fragrant oil, such as Laotian flower essential oil, for 2-5 minutes. The selected fragrant oil may target the person of an element type.

Step 3: (Elimination Therapy) Drink selected tea, such as Laotian lemongrass tea, to help eliminate asbestos from the human body through urination. A resonance-based test technique, such as the BDORT technique, may be used to measure and record the amount of asbestos in the urine before and after practicing the procedure.

The procedure may be practiced in a select order. Practicing the procedure may further comprise removing or excreting other toxins, germs, gene mutation cells, and cancer stem cells from the human body to urine, mucus, feces, or the skin and body surface.

FIGS. 11A to 11B show an embodiment of practicing a Hand and Foot Wash procedure to remove mercury from the human body before, after or during a music therapy, in accordance with the invention. The Hand and Foot Wash procedure may comprise the following one or more steps:

Step 1: Boil one packet of Deegua Root (100 gram) with 18,000 cc water; let it cool down; pour 1,000 cc water each time in a washbasin for washing hands and feet; and spray SF 93 Meridian water 40 or more times into the washbasin just before use.

Step 2: In accordance with FIG. 11A, use one hand to scoop up specially processed herbal water, such as Deegua Root water, to wash the other hand and forearm; pour down water and wash along the inside of forearm starting from the arm creases opposite the elbow down to the finger tips and then from the outside of finger tips up to the elbow completing one cycle; 40 or more cycles may be required.

Step 3: Switch to another hand and repeat Step 2.

Step 4: In accordance with FIG. 11B, put one foot in a washbasin; start washing from the big toe side and move to the back and along the leg up to the inner side (medial side) of the knee and then from the outer side (lateral side) of the knee down to the little toe to complete one cycle; 40 or more cycles may be required.

Step 5: Switch to another leg and repeat Step 4; if the washbasin is big enough, then proceed washing with both legs simultaneously.

The procedure may be practiced in a select order. Practicing the procedure may further comprise removing or excreting other toxins, germs, gene mutation cells, and cancer stem cells from the human body to urine, mucus, feces, or the skin and body surface.

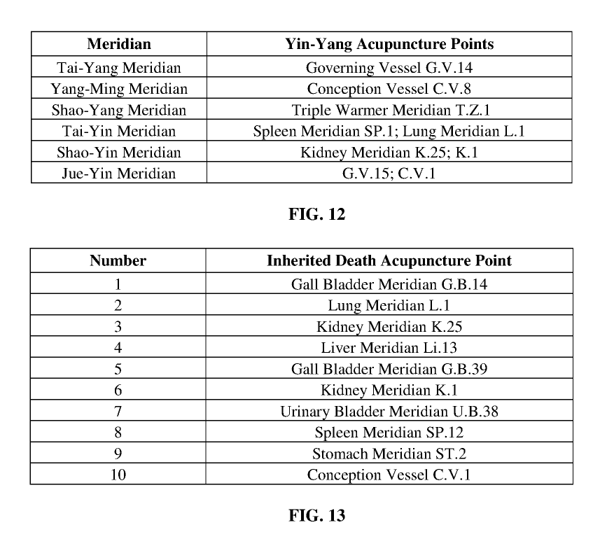

FIG. 12 shows an embodiment of practicing a first Herbal Water Spray procedure to remove mercury from any of the 6 or more Yin-Yang Acupuncture Points in the six layers in the meridian system of the human body before, after or during a music therapy, in accordance with the invention. The Triple Warmer Meridian shown in FIG. 12 is also known as Triple Burner Meridian or Triple Heater Meridian. The specially processed herbal water may be any kind of sulfhydryl group (SH)-containing herbs that exhibit high binding affinity of mercury, such as SF 93 Meridian water. The six outermost to innermost layers in the meridian system are Tai-Yang Meridian (associated with the skin and body surface), Yang-Ming Meridian (associated with the gastrointestinal tract, or GI tract), Shao-Yang Meridian (associated with the lymphatic system), Tai-Yin Meridian (associated with spleen and lung), Shao-Yin Meridian (associated with heart and kidney), and Jue-Yin Meridian (associated with brain and liver). The Yin-Yang Acupuncture Points may detect the presence or absence of mercury in each layer. The locations of these Yin-Yang Acupuncture Points may be found in any Acupuncture textbook.

The first Herbal Water Spray procedure may further comprise using the following one or more steps to remove or excrete mercury from the human body:

Step 1: Use Deegua Root water to wash hands and feet to remove mercury from the skin and body surface.

Step 2: Eat cooked yam leaf to remove mercury from the gastrointestinal tract.

Step 3: Use exercise, acupuncture, moxibustion, or traditional massage to remove mercury from the lymphatic system.

Step 4: Boil common selfheal fruit-spike or peeled ginger and inhale the steam when it is not too hot to remove mercury through perspiration.

Step 5: Take Sirtwin, lemongrass, or Coocha tree to remove mercury through urination.

Step 6: Spray SF 93 Meridian water 40 or more times each on Governing Vessel G.V.15 and Conception Vessel C.V.1, or Liver Meridian Li.14 and Liver Meridian Li.1, to remove mercury from these acupuncture points. Forty or more times each on the two spots is one cycle; spray more cycles until there is zero detection of mercury in these acupuncture points. The step may require one or more cycles to ensure that there is no mercury resonance detectable.

The procedure may be practiced in a select order. Practicing the procedure may further comprise removing or excreting other toxins, germs, gene mutation cells, and cancer stem cells from the human body to urine, mucus, feces, or the skin and body surface.

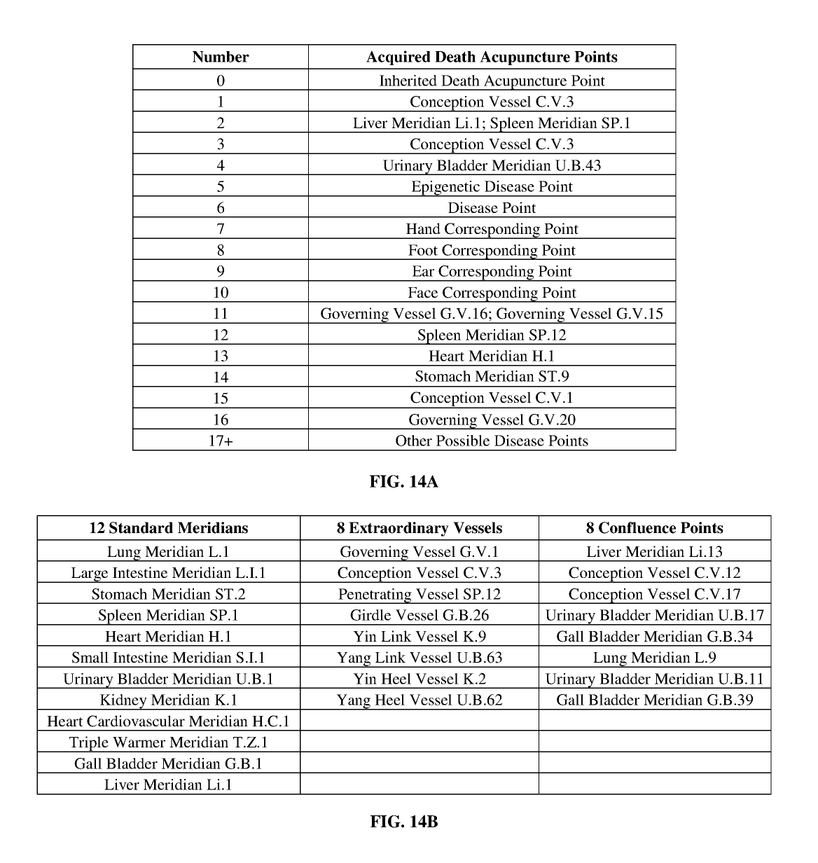

FIG. 13 shows an embodiment of practicing a second Herbal Water Spray procedure to remove mercury from any of the 10 Inherited Death Acupuncture Points in the human body before, after or during a music therapy, in accordance with the invention. Every person exhibits one of the 10 Inherited Death Acupuncture Points which are the starting locations that may emit the resonance information waves of asbestos, mercury, cancer stem cells, 80,000-gauss EMF, etc. This specific acupuncture point is inherited from either parent. The Inherited Death Acupuncture Point becomes the starting point of a Death Information Wave. The Death Information Wave is an electromagnetic wave that travels from a starting point through some intermediate points to the end point. The procedure may comprise spraying the specially processed herbal water, such as SF 93 Meridian water, on any of the 10 Inherited Death Acupuncture Points in the human body as shown in FIG. 13. The locations of these Inherited Death Acupuncture Points may be found in any Acupuncture textbook. Practicing the procedure may further comprise removing or excreting other toxins, germs, gene mutation cells, and cancer stem cells from the human body to urine, mucus, feces, or the skin and body surface.

FIGS. 14A and 14B show an embodiment of practicing a third Herbal Water Spray procedure to remove mercury from any of the 16 or more Acquired Death Acupuncture Points in the human body before, after or during a music therapy, in accordance with the invention. The specially processed herbal water may be any kind of sulfhydryl group (SH)-containing herbs that exhibit high binding affinity of mercury, such as SF 93 Meridian water. The procedure may comprise spraying the specially processed herbal water on 16 or more Acquired Death Acupuncture Points in the human body as shown in FIG. 14A.

Spraying the specially processed herbal water 40 or more times on the Death Acupuncture Point may cause the resonance of a Death Information Wave to recede along the meridian system. In other words, the resonance of the Death Information Wave would make a sequential retreat from the starting point back to the source (the end point). The Inherited Death Acupuncture Point and the 16 Acquired Death Acupuncture Points exhibit the resonance of the Death Information Wave sequentially. On the human body, despite the starting point (the 0^{th} Inherited Death Acupuncture Point), the following 1^{st} through 4^{th} locations are the same, but the 5^{th} through 10^{th} locations are different and are decided by the types of illness the person may experience. A resonance-based test technique, such as the BDORT technique, may be used to find these locations. The 5^{th} location, referred to as the Epigenetic Disease Point, is found by searching through the origins of the 12 standard meridians, 8 extraordinary vessels, and 8 confluence points in that order as shown in FIG. 14B. The 6^{th} location is found by searching through the related anatomical positions. The Heart Cardiovascular Meridian is also known as Pericardium Meridian or Heart Protector Meridian. Therefore, the 5^{th} through 10^{th} locations provide clues for the differential diagnosis. The 11^{th} through 16^{th} locations are the same for each person again. The end point (the source) of the Death Information Wave is G.V.20. The additional 17^{th}-plus locations are anatomical positions again; they are different and are decided whether the person has other illnesses. The locations of these Acquired Death Acupuncture Points may be found in any Acupuncture textbook and (Omura 2012). Practicing the procedure may further comprise removing or excreting other toxins, germs, gene mutation cells, and cancer stem cells from the human body to urine, mucus, feces, or the skin and body surface.

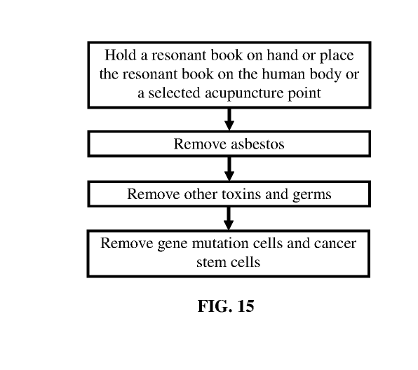

FIG. 15 shows an embodiment of using a resonant book to remove or excrete asbestos from the human body, in accordance with the invention. The person may hold a resonant book on hand or place the resonant book on a selected acupuncture point or on the human body, with or without practicing the music therapy. The resonant book, referred to as an optimal-dose resonant book, may comprise one or more books which can cause resonance with the human body when using a resonance-based test technique (such as BDORT). The book may comprise a religious, science or medicine book (such as a book of the Bible). The selected acupuncture point may be Conception Vessel C.V.8. Practicing the procedure may further comprise removing or excreting other toxins, germs, gene mutation cells, and cancer stem cells from the human body to urine, mucus, feces, or the skin and body surface.

Having thus described and illustrated specific embodiments of the invention, it is to be understood that the objectives of the invention have been fully achieved. And it will be understood by those skilled in the art that many changes in construction and circuitry, and widely differing embodiments and applications of the invention will suggest themselves without departing from the spirit and scope of the invention. The disclosures and the description herein are intended to be illustrative and are not in any sense limitation of the invention, more preferably defined in scope by the following claims.

What is claimed is:

1. An apparatus for removing or excreting asbestos from a human body using a music therapy, the music therapy comprising a set of one or more vibrational energy songs, the apparatus comprising (a) storing said vibrational energy songs on a music player, cell phone or computer, and (b) listening to said vibrational energy songs on said music player, cell phone or computer, wherein said vibrational energy song further comprises three or more notes solely using at most six of the following seven music notes: {Do (1), Re (2), Mi (3), Fa (4), Sol (5), La (6), Si (7)}.

2. The apparatus of claim 1, wherein said note is sung or played at a higher pitch or a lower pitch.

3. The apparatus of claim 1, wherein said music therapy further comprises appending {| 1+2+5 6 |} to the end of said set of two to five vibrational energy songs, wherein said 1+ is a higher pitch of Do (1), said 2+ is a higher pitch of Re (2), and said “|” indicates the end of the previous note or syllable.

4. The apparatus of claim 1, wherein said vibrational energy song further comprises {| 1+2+3+ | 3+5 3 | 3 1 2 | 2 3 5 | 5 3 1+|}, wherein said 1+ is a higher pitch of Do (1), said 2+ is a higher pitch of Re (2), said 3+ is a higher pitch of Mi (3), and said “| ” indicates the end of the previous note or syllable.

5. The apparatus of claim 1, wherein said vibrational energy song further comprises {| 2 3 5 6 | 6 3+2+3+ | 3+5 6 3+ | 3+2+3+5 | 5 5 6 3 |}, wherein said 2+ is a higher pitch of Re (2), said 3+ is a higher pitch of Mi (3), and said “|” indicates the end of the previous note or syllable.

6. The apparatus of claim 1, wherein said vibrational energy song further comprises {| 1 3 5 6 | 6 3+1+3+ | 3+5 6 3 | 3 1 3 5 | 5 6 3 1+|}, wherein said 1+ is a higher pitch of Do (1), said 3+ is a higher pitch of Mi (3), and said “|” indicates the end of the previous note or syllable.

7. The apparatus of claim 1, wherein said vibrational energy song further comprises {| 1 2 5 6 | 6 1+2+5 | 5 6 1+2+ | 2+5 6 1+|}, wherein said 1+ is a higher pitch of Do (1), said 2+ is a higher pitch of Re (2), and said “|” indicates the end of the previous note or syllable.

8. The apparatus of claim 1, wherein said vibrational energy song further comprises {| 1 2 3 | 3 6 3+| 3+1+2+| 2+3+6 | 6 3+1+|}, wherein said 1+ is a higher pitch of Do (1), said 2+ is a higher pitch of Re (2), said 3+ is a higher pitch of Mi (3), and said “| ” indicates the end of the previous note or syllable.

9. The apparatus of claim 1, further comprising clapping hands or pressing corresponding acupuncture points of an internal organ on one hand or both hands during said music therapy.

10. The apparatus of claim 1, wherein said music therapy further comprises spraying specially processed herbal water on a selected acupuncture point before or after said music therapy.

11. The apparatus of claim 1, wherein said music therapy further comprises removing or excreting mercury, lead, arsenic, aluminum, viruses, bacteria, fungi, or any combination of the above, from the human body to urine, mucus, feces, or the skin and body surface.

12. The apparatus of claim 1, wherein said music therapy further comprises removing or excreting CD332, CD24, CD44, CD133, CD29, CD152, or any combination of the above, from the human body to urine, mucus, feces, or the skin and body surface.

13. The apparatus of claim 1, wherein said music therapy further comprises placing said set of one or more vibrational energy songs in a select order, or repeating said music therapy as many times as is desired at any time.

14. An apparatus for removing or excreting asbestos from a human body or reducing insomnia of a person using a music therapy, the music therapy comprising a set of one or more vibrational energy songs, the apparatus comprising (a) storing said vibrational energy songs on a music player, cell phone or computer, and (b) listening to said vibrational energy songs on said music player, cell phone or computer, wherein said vibrational energy song further comprises three or more notes solely using at most six of the following seven music notes: {Do (1), Re (2), Mi (3), Fa (4), Sol (5), La (6), Si (7)}.

15. The apparatus of claim 14, wherein said note is sung or played at a higher pitch or a lower pitch.

16. The apparatus of claim 14, wherein said music therapy further comprises appending {| 1+2+5 6 |} to the end of said set of two to five vibrational energy songs, wherein said 1+ is one pitch higher than Do (1), said 2+ is one pitch higher than Re (2), and said “|” indicates the end of the previous note or syllable.

17. The apparatus of claim 14, wherein said vibrational energy song further comprises {| 6++3+++6++| 1+++2++6+ | 6+3+6+| 1+2+6 | 6 3 6 |}, wherein a note N+ is one pitch higher than N; N++ is one pitch higher than N+; and N+++ is one pitch higher than N++, and said “|” indicates the end of the previous note or syllable.

18. The apparatus of claim 14, wherein said vibrational energy song further comprises {| 3++5++6++| 6++2+++6++| 3++5+6+| 6+2+6+| 3 5 6 |}, wherein a note N+ is one pitch higher than N; N++ is one pitch higher than N+; and N+++ is one pitch higher than N++, and said “|” indicates the end of the previous note or syllable.

19. The apparatus of claim 14, wherein said vibrational energy song further comprises {| 6 1+6 I 6+3+5+| 6+1++6+| 6++3++5++| 6++1++1+++|}, wherein a note N+ is one pitch higher than N; N++ is one pitch higher than N+; and N+++ is one pitch higher than N++, and said “|” indicates the end of the previous note or syllable.

20. The apparatus of claim 14, wherein said vibrational energy song further comprises {| 1 2 3 | 3 5 1+| 1+2+3+| 3+5+1++| 1++2++3++|}, wherein a note N+ is one pitch higher than N; and N++ is one pitch higher than N+, and said “| ” indicates the end of the previous note or syllable.

21. The apparatus of claim 14, wherein said vibrational energy song further comprises {| 1 2 6− | 6 5+6+| 1++2++6+| 6++5++6++| 1+++2+++6++|}, wherein a note N is one pitch higher than N−; N+ is one pitch higher than N; N++ is one pitch higher than N+; and N+++ is one pitch higher than N++, and said “|” indicates the end of the previous note or syllable.

22. The apparatus of claim 14, further comprising clapping hands or pressing corresponding acupuncture points of an internal organ on one hand or both hands during said music therapy.

23. The apparatus of claim 14, wherein said music therapy further comprises spraying specially processed herbal water on a selected acupuncture point before or after said music therapy.

24. The apparatus of claim 14, wherein said music therapy further comprises removing or excreting mercury, lead, arsenic, aluminum, viruses, bacteria, fungi, or any combination of the above, from the human body to urine, mucus, feces, or the skin and body surface.

25. The apparatus of claim 14, wherein said music therapy further comprises removing or excreting CD332, CD24, CD44, CD133, CD29, CD152, or any combination of the above, from the human body to urine, mucus, feces, or the skin and body surface.

26. The apparatus of claim 14, wherein said music therapy further comprises placing said set of one or more vibrational energy songs in a select order, or repeating said music therapy as many times as is desired at any time.

27. An apparatus for removing or excreting asbestos from a human body using a resonant book, the apparatus comprising (a) holding a resonant book on hand or (b) placing the resonant book on the human body or a selected acupuncture point, wherein said resonant book further comprises one or more books which can cause resonance with the human body.

28. The apparatus of claim 27, wherein said one or more books further comprises a religious, science or medicine book.

29. The apparatus of claim 27, wherein said resonant book further comprises removing or excreting mercury, lead, arsenic, aluminum, viruses, bacteria, fungi, or any combination of the above, from the human body to urine, mucus, feces, or the skin and body surface.

30. The apparatus of claim 27, wherein said resonant book further comprises removing or excreting CD332, CD24, CD44, CD133, CD29, CD152, or any combination of the above, from the human body to urine, mucus, feces, or the skin and body surface.
